(19) United States
(12) Patent Application Publication
(10) Pub. No.: US 2025/0360204 A1
(43) Pub. Date: Nov. 27, 2025

(54) ADJUVANT COMPOSITION CONTAINING GALACTAN STRUCTURE AND USES THEREOF

(71) Applicant: Seoul National University R&DB Foundation, Seoul (KR)

(72) Inventors: SungWon Kwon, Seoul (KR); GyuHwan Hyun, Seoul (KR); SeulJi Lee, Seoul (KR)

(21) Appl. No.: 18/737,544

(22) Filed: Jun. 7, 2024

(30) Foreign Application Priority Data
May 21, 2024 (KR) 10-2024-0066095

(51) Int. Cl. A61K 39/39; A61K 39/00; C12P 19/04

(52) U.S. Cl. CPC A61K 39/39; C12P 19/04; A61K 2039/55583

(57) ABSTRACT

The present invention relates to an adjuvant composition including a galactose polymer which is interconnected by β-1,4 bonds. The adjuvant composition of the present invention is a novel immunoactive adjuvant including an immunoactive structure having a galactan repeating sugar structure that has not been previously identified, and it has different mechanisms of action and operating principles compared to existing adjuvants. The adjuvant composition of the present invention has a structure based on plant natural polysaccharides, and it has high economic feasibility due to the characteristics of the carbohydrate structure, is biodegradable and thus environmentally friendly, and has high chemical stability. In addition, the adjuvant composition of the present invention has the advantage of increasing anti-cancer efficacy when used in combination with existing immunotherapy drugs as an immune checkpoint inhibitor by activating tumor-associated macrophages in a tumor microenvironment, and particularly has the advantage of excellent efficacy on tumors with low immune response.

### CROSS-REFERENCE TO RELATED APPLICATION

This application claims priority to and the benefit of Korean Patent Application No. 10-2024-0066095, filed on May 21, 2024, the disclosure of which is incorporated herein by reference in its entirety.

### TECHNICAL FIELD

The present invention relates to an adjuvant composition including a galactan structure and uses thereof.

### BACKGROUND ART

Carbohydrates are the most abundant type of biomolecule on Earth. Carbohydrate-based biopolymers have traditionally been used to develop inert matrices or support materials, such as cellulose or dextran derivatives. However, due to the structural diversity thereof, there are methods to develop active substances that induce biological responses. Therefore, bioactive carbohydrates remain an untapped source of functional foods and therapeutics. Complex carbohydrates can act as natural supramolecular immunomodulators, because the multiplicity of glycan residues promotes innate immune recognition.

Unlike chemotherapy, which directly acts on tumor cells, immunotherapy is a cutting-edge strategy that indirectly attacks tumor cells by activating the body's immune system to induce anti-cancer efficacy. This is a very effective and groundbreaking method, but the efficacy thereof varies greatly depending on the characteristics of each tumor tissue, and thus, there is a disadvantage in that the clinical response varies greatly from patient to patient.

The tumor microenvironment (TME) is a major factor that determines the efficacy of immunotherapy, and the tumor microenvironment refers to the totality of physiological components such as immune cells, peripheral cells and neovascularization around tumor cells. In other words, it determines the reactivity and characteristics of tumor tissue. If there are many immune suppressor cells in the tumor microenvironment, it causes a cold tumor with low immune response, which lowers the efficacy of immune activation by immunotherapy and makes it difficult for immune cells to penetrate into tumor tissue, thereby ultimately evading the immune system. Therefore, a strategy to remodel the tumor microenvironment to make immunotherapy effective is attracting attention.

The efficacy of immunotherapy can be enhanced through the re-education of tumor-associated macrophages (TAMs). Tumor-associated macrophages (TAMs) in the tumor microenvironment play a key role in determining the immune environment of the tumor, and are thus becoming a key target of the tumor microenvironment remodeling strategy. Therefore, by re-educating tumor-associated macrophages through appropriate immunological stimulation, the immunosuppressive tumor microenvironment can be remodeled, thereby improving the immune response of tumors. These immunological stimulants can be administered in combination with immunotherapy agents to effectively induce anti-cancer activity in tumors that could not be expected to have sufficient anti-cancer effects with existing immunotherapy.

As the strategy of remodeling the tumor microenvironment for improving immunotherapy has been gaining attention, various types of immunomodulators have been developed. Among these stimulants that are being applied in research include small molecules, bacterial-ligand-mimetic agents, antibodies, microRNA and attenuated or bioengineered live bacteria. However, previously developed immunomodulators have inherent disadvantages. Small molecule stimulants exhibit non-target effects and low biocompatibility, and many immunostimulants derived from bacterial lipid structures exhibit non-specific immune activity and low solubility. Proteins, cytokines, antibodies and RNA have biocompatibility, but they have the disadvantages of having low stability, high cost and unpredictable immunogenicity. In order to minimize these problems, many innovative drug delivery systems and new formulations have been developed, but it is difficult to solve the fundamental problems of the active substance itself. Therefore, the development of a medicament having a new structure with greater efficacy and safety is required.

Accordingly, the inventors of the present invention have completed the present invention as a strategy to remodel the tumor microenvironment by using an immunomodulatory adjuvant that re-educates tumor-associated macrophages existing in the tumor in order to improve the low responsiveness of cancer immunotherapy in immune-hyporesponsive tumors, by focusing on natural carbohydrates with high immunomodulatory ability.

### RELATED ART DOCUMENTS

### Patent Documents

Korean Patent Application Laid-Open No. 10-2017-0139325 (Dec. 19, 2017)

### DISCLOSURE

### Technical Problem

An object of the present invention is to provide an adjuvant composition, including a galactose polymer which is interconnected by β-1,4 bonds.

Another object of the present invention is to provide a vaccine composition, including the adjuvant composition.

Still another object of the present invention is to provide a method for preparing an adjuvant composition, including the steps of extracting pectin heteropolysaccharide (PHP); and sequentially treating the extracted pectin heteropolysaccharide (PHP) with enzymes.

### Technical Solution

The present invention provides an adjuvant composition, including a galactose polymer which is interconnected by β-1,4 bonds.

In addition, the present invention provides a vaccine composition, including the adjuvant composition.

In addition, the present invention provides a method for preparing an adjuvant composition, including the steps of extracting pectin heteropolysaccharide (PHP); and sequentially treating the extracted pectin heteropolysaccharide (PHP) with enzymes.

### Advantageous Effects

The present invention relates to an adjuvant composition including a galactose polymer which is interconnected by β-1,4 bonds. The adjuvant composition of the present invention is a novel immunoactive adjuvant including an immunoactive structure having a galactan repeating sugar structure that has not been previously identified, and it has different mechanisms of action and operating principles compared to existing adjuvants. The adjuvant composition of the present invention has a structure based on plant natural polysaccharides, and it has high economic feasibility due to the characteristics of the carbohydrate structure, is biodegradable and thus environmentally friendly, and has high chemical stability. In addition, the adjuvant composition of the present invention has the advantage of increasing anti-cancer efficacy when used in combination with existing immunotherapy drugs as an immune checkpoint inhibitor by activating tumor-associated macrophages in a tumor microenvironment, and particularly has the advantage of excellent efficacy on tumors with low immune response.

### DESCRIPTION OF DRAWINGS

FIG. 1 shows the design of a carbohydrate immunomodulatory anti-cancer adjuvant construct (Gal-NC). a) Structural representation of the glycosyl branch of pectic heteropolysaccharides (PHP) and activity screening using carbohydrate-active enzymes (CAZymes) (n=5). b) Brief procedure for preparation of Gal-NC by enzymatic transformation to enrich multiple galactan glycopatterns. c) Visualization of Gal-NC particle formation using transmission electron microscopy (TEM) (scale bar: 100 nm).

FIG. 2 shows the preparation and characterization of carbohydrate immunomodulatory anti-cancer adjuvant constructs. a) Separation of Gal-NC from enzymatic reaction product using preparative size exclusion chromatography (Prep-SEC). b) Macrophage stimulating efficacy of Gal-NC compared to fragments (n=3). c) Physicochemical properties of Gal-NC. Gal-NC has a ζ potential of −25 mV and a diameter of 140 nm as assessed by dynamic light scattering (DLS). Gal-NC consists of repeating galactans that are attached to a rhamnogalacturonan backbone (scale bars: 100 nm, white; 50 nm, black). d) Structural enrichment of galactose oligosaccharide chains in Gal-NC. The degree of polymerization (DP) of the galactan branches was approximately 10. e) Molecular weight measurement of Gal-NC chains using multi-angle light scattering coupled with size exclusion chromatography (SEC-MALS). f) Small-angle X-ray scattering (SAXS) profile of diluted Gal-NC. g) Fluorescence image showing Gal-NC attached to the surface of macrophages (scale bar: 100 m). *p<0.05, **p<0.01, ***p<0.001, ****p<0.0001.

FIG. 3 shows the results of receptor binding simulation and receptor-complex formation confirmation of carbohydrate immunomodulatory anti-cancer adjuvant constructs. a) In silico reverse docking screening for possible target receptors. It is a heatmap showing docking affinity of 6 galactose oligosaccharides from Gal4 to Gal9 with the crystal structures of selected receptors. The top 10 docking structures are indicated by preference for each combination. b) Best docking position between Gal9 and the structure of the mTLR4/MD2 complex and 3VQ2. c) Distance distribution between the C-terminus of the LRR domain of 3VQ2 and the ligand in the molecular dynamics simulation trajectory for 100 ns at 300 K. d) rTLR4-Gal-NC complex separated by SEC.

FIG. 4 shows the multivalent binding behavior of carbohydrate immunomodulatory anti-cancer adjuvant constructs. a) Sensorgrams for rTLR4-immobilized surfaces (left: Gal-NC, right: lipopolysaccharide [LPS]). b) Dose-response curves of Gal-NC and other TLR ligands using murine TLR4 and TLR2 reporter HEK293 cells. c) Co-localization of TLR4 and Gal-NC observed upon fluorescent staining of BMDMs using Cy3-tagged Gal-NC (Cy3-Gal-NC) and FITC-tagged TLR4 antibody (FITC-TLR4). Intensity profiles (bottom right) show the overlapping distribution of FITC and Cy3 along the axis that passes through the continuous aggregates within the white-lined ROI (scale bars: 5 m, left; 2 m, right). d) Schematic illustration of the expected binding behavior of Gal-NC that induces the formation of TLR4 clusters on the macrophage surface.

FIG. 5 shows the results of immunostimulatory signaling pathway activation assays in macrophage models of carbohydrate immunomodulatory anti-cancer adjuvant constructs. a) Volcano plot showing differentially expressed gene (DEG) patterns in Gal-NC-treated macrophages. b) Enrichment plot showing upregulated pathways in Gal-NC treated BMDM (obtained using GSEA). b) Cytotoxic effect on macrophages after Gal-NC treatment. c) Venn diagram of DEGs including gene sets from Gal-NC (experimental) and LPS (GSE1155801)-treated BMDM obtained from GEO. d) Heatmap showing immune-related pathways enriched in Gal-NC treated or LPS-stimulated BMDM. e) Gal-NC treatment showed a response pattern similar to LPS-mediated TLR signaling pathway in BMDM. f) NO secretion by Gal-NC-mediated TLR4 activation was reduced by pretreatment with TLR4 inhibitor (n=3). *p<0.05, **p<0.01, ***p<0.001, ****p<0.0001.

FIG. 6 shows the results of biochemical analysis to determine the macrophage re-education efficacy of carbohydrate immunomodulatory anti-cancer adjuvant constructs. a) Macrophage re-education process by Gal-NC treatment. Distinct morphological changes were observed during macrophage polarization. b) Representative FACS plots of CD86-CD206+ or CD86+CD206− upon Gal-NC treatment. c) Changes in CD86-CD206+ and CD86+CD206− populations as a function of Gal-NC treatment concentration. Shown as mean±SD. **p<0.01, ***p<0.001 versus M(IL-4). d) qPCR analysis for changes in induced mRNA expression of M(IL-4) after Gal-NC treatment. Shown as mean±SD. e-f) Measurement of the levels of TNF-α (e) and IL-12p70 (f) secreted by Gal-NC-induced macrophages. n=3. Shown as mean±SD. *p<0.05, **p<0.01, ***p<0.001, ****p<0.0001 versus PBS control. g) Heatmap showing changes in polyunsaturated fatty acids (PUFA) and their derivatives secreted by macrophages according to Gal-NC treatment.

FIG. 7 shows the results of macrophage-dependent anti-tumor effects of carbohydrate immunomodulatory anti-cancer adjuvant constructs. a) MC38 inoculation tumor growth model in C57BL/6 mice. b) Tumor growth curves up to 20 days after injection of PBS and Gal-NC treatment in naïve or macrophage-depleted mice. n=10 for each group (mean±SEM, difference=−1,187±309.6 mm3, **p=0.0012, two-tailed unpaired t-test). c) Representative images of tumors from PBS- and Gal-NC-treated mice. d) Overall view of MC38 tumors and tumor weights harvested on day 20 (mean±SEM, **p=0.0066, two-tailed unpaired t-test). e) The proportion of TAMs (F4/80+CD11b+) among CD45+immune cells in PBS- and Gal-NC-treated tumor tissues is shown (top). The proportions of M1-like TAMs (CD11b+F4/80+CD86+) and M2-like TAMs (CD11b+F4/80+CD206+) are shown (bottom) (mean±SD, *p=0.0228, **p=0.0009, two-tailed unpaired t-test). f) Immunohistochemically stained MC38 tumor tissue images showing CD206+ (red) and Ly6C+ (green) cells. Nuclei were counterstained with DAPI (blue) (scale bar: 100 m). g) Near-infrared fluorescence (NIRF) images used to measure the distribution of CD206+ macrophages in MC38 tumors. CD206 was targeted by the intraperitoneal injection of cyanine-7-tagged mannose (Man-Cy7).

FIG. 8 shows the results of the synergistic effect analysis by the immunotherapy adjuvant construct. a) Phenotypic changes of T cells in MC38 tumors. The leukocyte population was controlled by FSC and SSC. After excluding dead cells, T cell subsets were gated based on the surface expression of CD45, CD3, CD4, CD8 and Foxp3 (mean±SD, *p=0.0158 CD3+CD8+ of CD45+; *p=0.0434 Foxp3+ of CD3+CD4+, two-tailed unpaired t-test). b) Schematic diagram of animal experiments to investigate the association of Gal-NC's anti-tumor effect and adaptive immunity in panels c-e. c) Representative images of MC38 tumors in nude mice. d) Tumor growth curves and e) tumor volumes on day 16. Mean±SEM values are shown. f) Experimental design to investigate the synergistic effect of Gal-NC and αPD-1 monoclonal antibody (mAb). g) Tumor volume in MC38 and B16-F10 tumor growth models on day 8 of co-administration (n=7, mean±SEM, ***p=0.0007, compared with PBS control using Brown-Forsythe and Welch ANOVA test with Dunnett's T3 multiple comparisons). h) Change in tumor size over day 8 compared with the initial tumor size on day 0. i) Survival period after treatment initiation (*p=0.468 Gal-NC in MC38; ***p=0.0002 Gal-NC+αPD-1 in MC38; *p=0.0296 Gal-NC in B16-F10; ****p<0.0001 Gal-NC+αPD-1 in B16-F10 compared with PBS control, log-rank Mantel-Cox test). j) Analysis in B16-F10 tumors of 4 groups (mean±SD, one-way ANOVA with Dunnett's multiple comparisons). *p<0.05, **p<0.01, ***p<0.001.

FIG. 9 shows that the carbohydrate immunomodulatory anti-cancer adjuvant construct enhanced cancer immunotherapy by remodeling TME. a) Multivalent pattern recognition of Gal-NC by TLR induces NF-κB activation in macrophages. b) Signaling cascades triggered by Gal-NC effectively reprogram immunosuppressive TAMs into immune-enhancing macrophages. c) Phenotypic transition of TAM populations immunologically remodels TME with immunocompetent characteristics, thereby enhancing the therapeutic efficacy of ICB.

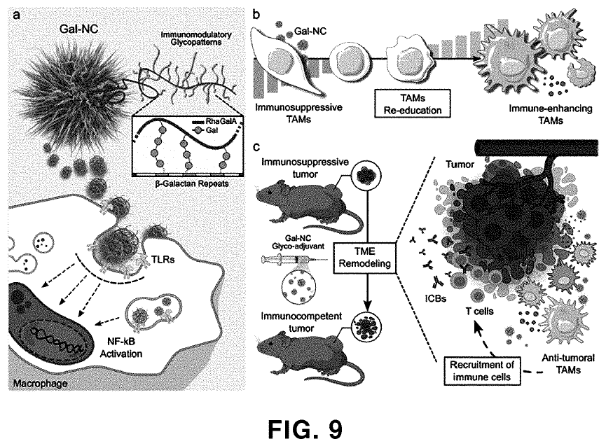

### MODES OF THE INVENTION

Current cancer immunotherapy strategies mainly focus on remodeling the tumor microenvironment (TME) to favor anti-tumor immunity. Increasing attention is being paid to developing innovative immunomodulatory adjuvants that can restore weakened anti-tumor immunity by imparting immunogenicity to inflamed tumor tissues.

The inventors of the present invention have completed a galactose polymer (Gal-NC) that favorably responds to the host immune system in the tumor microenvironment (TME). The galactose polymer treatment in the present invention induced a synergistic anti-tumor effect in combination with αPD-1 mAb. As the regulation of the immune system has become increasingly important in cancer treatment, the galactose polymer according to the present invention provides a potential therapeutic strategy that can enhance immunogenicity and improve the efficacy of T cell-based therapy.

Hereinafter, the present invention will be described in more detail.

The present invention provides an adjuvant composition, including a galactose polymer which is interconnected by β-1,4 bonds.

The inventors of the present invention have completed galactose polymers (Gal-NC) that are interconnected by β-1,4 bonds as a structure rich in galactan, which is a polysaccharide. According to one embodiment of the present invention, the galactose polymer is derived from a natural carbohydrate structure through an enzymatic conversion optimized for effective, stable and biologically safe innate immune regulation. In addition, the galactose polymer is negatively charged, and the structural assembly is spontaneously aggregated at physiological pH (about 7) through intermolecular electrostatic forces, and it was confirmed that it showed a weak anionic characteristic with a zeta potential of about −25 mV.

In the present invention, the galactose polymer may be recognized by macrophages and induce TLR4-specific activation. The galactan repeat structure in the galactose polymer functions as a multivalent pattern recognition site for Toll-like receptor 4 (TLR4). Functionally, galactose polymer-mediated TLR activation induces the repolarization of tumor-associated macrophages (TAMs) toward an immune-stimulating/tumor-removing M1-like phenotype. The galactose polymer increases the intratumoral population of cytotoxic T cells, which are major effector cells of anti-tumor immunity, through reeducated TAMs.

In the present invention, the galactose polymer may be specific for the innate immune signaling pathway.

In the present invention, the galactose polymer may reprogram M2-polarized macrophages into an M1-like phenotype in a TLR4-dependent manner.

In the present invention, the galactose polymer may change the tumor microenvironment (TME) by reducing the number of helper T cells in the tumor. The TME change synergistically enhances the T cell-mediated anti-tumor response induced by αPD-1 administration, which means that the galactose polymer has an adjuvant effect in immune checkpoint blockade combination therapy.

In the present invention, the galactose polymer may be a polymer in which 4 to 20 galactose units are linked.

In the present invention, the galactose polymer may have a molecular weight of 100,000 to 200,000 kDa.

In addition, the present invention provides a vaccine composition, including the adjuvant composition.

In the present invention, the vaccine composition may further include an antigen.

In the present invention, the antigen may be selected from the group consisting of proteins, cells, viruses and combinations thereof.

Since the features corresponding to the above-described adjuvant composition included in the above-described vaccine composition can be replaced in the above-described part, the description thereof will be omitted.

In addition, the present invention provides a method for preparing an adjuvant composition, including the steps of extracting pectin heteropolysaccharide (PHP); and sequentially treating the extracted pectin heteropolysaccharide (PUP) with enzymes.

In the present invention, the pectin heteropolysaccharide (PUP) may be extracted from ginseng (Panax ginseng).

In the present invention, Arabinase (ARA), endo-polygalacturonase (GALA) and amylase (AMY) may be sequentially treated as the enzymes.

Since the features corresponding to the above-described adjuvant composition included in the method for preparing the above-described adjuvant composition can be replaced in the above-described part, the description thereof will be omitted.

Hereinafter, in order to help understanding of the present invention, examples will be provided and described in detail. However, the following examples are only intended to illustrate the contents of the present invention, and the scope of the present invention is not limited to the following examples. The examples of the present invention are provided to more completely explain the present invention to a person having ordinary skill in the art.

### Example 1. Materials and Methods

### 1.1. Materials and Animals

All chemicals used in the experiment were purchased from Sigma-Aldrich (St. Louis, MO, USA) unless otherwise mentioned. Water, methanol, acetonitrile and other solvents including liquid chromatography (LC) eluents were purchased from J.T. Baker (Phillipsburg, NJ, USA). Biological reagents were purchased from Biocarbohydrate immunomodulatory anti-cancer adjuvant construct end (San Diego, CA, USA), eBioscience (San Diego, CA, USA) and Invitrogen (Waltham, MA, USA). Recombinant proteins were purchased from PeproTech (Cranbury, NJ, USA). All primers used for RT-qPCR were synthesized by Bioneer (Daejeon, South Korea). Mouse anti-PD-1 antibody was purchased from BioXcell (Lebanon, NH, USA). Antibodies used for immunoblotting analysis were as follows: anti-pERK (Cell Signaling, 9102, 1:1,000), anti-IκBα (Santa Cruz, 371, 1:1,000) and anti-p-p38 (Cell Signaling, 4511, 1):1,000).

Meanwhile, female C57BL/6 and BALB/c-nude mice (6 to 8 weeks old, 18 to 20 g) were approved by Seoul National University IACUC (SNU-210406-5-3; SNU-220117-10) and purchased from Koatech (Pyeongtaek, Korea).

### 1.2. Preparation of Raw Pectin Heteropolysaccharide (PUP)

Plant PUP was extracted from ginseng (Panax ginseng). When it is briefly explained, 200 g of dried ginseng root was finely ground and extracted three times by using 1 L of water in a boiling water bath at 80° C. The insoluble residue of the extract mixture was removed by centrifugation (10,000 g, 30 min), and the remaining extract was concentrated 10 times. Next, ice-cold ethanol was added four times, and the alcohol-insoluble residue (AIR) before protein removal was taken by using Sevag reagent to obtain the crude polysaccharide fraction. The crude polysaccharide fraction was completely dissolved in water and loaded onto a column with DEAE-Sepharose Fast Flow (Cytiva) anion exchange resin which was pre-equilibrated with water. After elution with 2 L of water, the acidic polysaccharide fraction (CW-AP) was eluted with 2 L of 0.5 M NaCl. Subsequently, CW-AP was reloaded onto the 11 DEAE-Sepharose columns by using a stepwise gradient of NaCl (0.0 M to 0.5 M). The intermediate anionic fraction, CW-AP-2, was obtained by elution with 0.2 M NaCl and subquantitatively separated by preparative size exclusion chromatography (Prep-SEC) by using a HiPrep 16/60 Sephacryl S-400 SEC column (Cytiva). Excess polysaccharide aggregates and fragments/salts were removed by Prep-SEC, and the active PUP fraction (50 kDa to 1,000 kDa) was purified.

### 1.3. Analysis of Monosaccharide Composition and Linkage in Raw Polysaccharides

Uronic acids of PHP were carboxylated to neutral sugars labeled with 6,6-dideutrium, respectively, and then converted to alditol acetate (AA) and partially methylated alditol acetate (PMAA) derivatives for monosaccharide and linkage analysis, respectively. PMAA derivatives were analyzed on a 7890A-5977B 12 GC-MS system (Agilent) equipped with a SP2380 column (100 m×0.25 mm, film thickness 0.20 m, Supleco). AA derivatives were also tested on an SP2380 column (30 m×0.25 mm, film thickness 0.20 m, Supelco) and a 7890A GC-FID system (Agilent). The relative monosaccharide compositions were obtained based on the FID responses. Uronic acids and their corresponding neutral sugars were distinguished by monitoring selected ions using GC-MS. Afterwards, the sugar linkages were then calculated based on the GC-MS data according to a previously reported method.

### 1.4. Enzyme Digestion Screening for Identification of Active Structures in Raw Polysaccharides

A panel of enzymes which are capable of cleaving specific glycan branches constituting heteropolysaccharides was constructed to screen for changes in activity due to structural disruption. When it is summarized, enzymes were prepared by redispersing each enzyme in a buffer solution at its optimal pH, and the complete cleavage activity was confirmed for each enzyme by using an azurin-crosslinked oligosaccharide substrate. PUP was prepared from a stock solution (10 mg/mL) and then spiked (100 μg/mL) into the enzyme dispersion which was dispensed at 200 μL to each well of a 96-well plate. Afterwards, the mixture was then sufficiently reacted at 40° C. for 1 day by using a thermoshaker. Subsequently, 100 μL of the reaction mixture was taken, and it was transferred to the RAW264.7 cell activity test plate. The release of nitric oxide (NO) induced from activated macrophages was assessed by using the Griess assay.

### 1.5. Preparation of Carbohydrate Immunomodulatory Anti-Cancer Adjuvant Structures Via Enzymatic Conversion of Heteropolysaccharides (PUP)

Arabinase (ARA), endo-polygalacturonase (GALA) and amylase (AMY) were first prepared at the appropriate working concentrations in appropriate buffers. The heteropolysaccharides were reconstituted in PBS at 1 mg/mL in a Teflon-capped reaction vial, and the debranching reaction was performed by sequentially adding the enzymes at 20 U/mL in a thermoshaker. The cleavage reactions of ARA, GALA and AMY were respectively performed at 40° C. for 20 hours, and the final step of the reaction was performed at 50° C. for 5 hours. In order to remove residual enzyme, the reaction mixture was deproteinized by using the Sevag method and then subjected to Prep-SEC using a HiPrep 16/60 Sephacryl S-400 SEC column (Cytiva) on an AKTA pure LC system (Cytiva). The high and low molecular weight fractions separated at 100 kDa were concentrated and lyophilized. The molecular weight distribution of both fractions was determined by using analytical SEC on an Ultrahydrogel 500 column (Waters) with a 1260 HPLC (Agilent) equipped with an 80 low-temperature evaporative light scattering detector (LT-ELSD). In order to confirm the activity, NO release from macrophages derived from each fraction was tested by using the Griess assay.

### 1.6. Analysis of Physical Properties of Carbohydrate Immunomodulatory Anti-Cancer Adjuvant Structure

A carbohydrate immunomodulatory anti-cancer adjuvant structure (1 g) was re-dispersed in 1 mL of PBS, sonicated, filtered (0.2 m) and serially diluted to a concentration of 0.01 μg/mL. The formation of a homogeneous carbohydrate immunomodulatory anti-cancer adjuvant structure solution was confirmed by the absence of Tyndall scattering. SAXS on the carbohydrate immunomodulatory anti-cancer adjuvant structure solution was performed by using Xeuss2.0 (Xenocs) with a PILATUS 300K detector (Dectris) at a wavelength of 1.54 Å for 1,800 s with a 1 mm capillary. The sample-to-detector distance was set to 2,500 mm. Size exclusion chromatography-multi-angle light scattering (SEC-MALS) was performed by using an LC-20AD HPLC system (Shimadzu) with a DAWN Heleos II light scattering detector (Wyatt) and an Optilab TRex Refractometer (Wyatt) at 25° C. SEC separation was performed on a TSK-gel-GMPWXL column at a flow rate of 0.5 mL/min with 300 mM NaCl buffer. The injection volume was set to 100 μL. Data analysis was performed by using ASTRA 6 software (Wyatt). The analysis of properties of particles was performed by using Zetasizer Pro (Malvern Panalytical) to determine the hydrodynamic size distribution of carbohydrate immunomodulatory anti-tumor adjuvant structure aggregates that were dispersed in phosphate buffer (pH 7.0) by dynamic light scattering (DLS) analysis. ζ-potentials were determined at 25° C. by using an ELS-Z1000 analyzer (Otsuka Electronics). For transmission electron microscopy (TEM), samples (1 g) were dispersed 15 mL in 1 mL buffer, spotted on Formvar/carbon film-coated 200 mesh TEM grids and rapidly dried before TEM imaging. TEM measurements were performed by using a LIBRA 120 energy-filtering transmission electron microscope (Carl Zeiss). ¹³C and ¹H nuclear magnetic resonance (NMR) spectra for structural characterization were recorded by using a JNM-ECA 600 MHz NMR spectrometer (JEOL). Polysaccharide samples (10 mg) were dissolved in D₂O (99.8%, 0.5 mL) and freeze-dried twice to completely remove residual H₂O. Afterwards, the dried samples were then redissolved in D₂O in a tube and introduced into the NMR spectrometer. All acquired spectra were analyzed by using MestReNva 14.0 software (Mestrelab Research). Fourier transform infrared (FT-IR) spectra of the samples were acquired by using an FT-IR-4200 spectrometer (JASCO) in the range of 4,000-600 cm⁻¹. Samples were measured on films of KBr disks. Data processing was performed by using Spectra Manager software (JASCO).

### 1.7. Analysis of Lipopolysaccharide (LPS) Contamination

An analysis was performed on bacterial LPS that could be incorporated into the plant polysaccharide extraction process. First of all, 3 reference LPS standards (Sigma) and a carbohydrate immunomodulatory anti-cancer adjuvant construct were weighed (0.3 mg each were redispersed in endotoxinfree water). The solutions were completely dried under nitrogen and redispersed in 400 L methanol. Afterwards, the sample was then directly introduced into methanolysis by adding 100 μL of 3 M methanolic HCl at 80° C. for 20 hours. After methanolysis, free fatty acids were extracted twice with n-hexane (1 mL). The collected hexane phase was evaporated under a stream of nitrogen before acetylation by adding pyridine (100 μL) and acetic anhydride (100 μL) at 100° C. for 1 hour. The sample was evaporated, redistributed in acetone (50 μL) and analyzed by using GC-MS QP2010 (Shimadzu). 3-Hydroxyl myristic acid (3-OH MA), which is a chemical marker of bacterial LPS, was detected by the retention time and the specific mass peak (m/z 257) of 3-OH MA standard (Sigma).

### 1.8. Inverse Docking and Molecular Dynamics (MD) Simulation for Target Protein Prediction

The target proteins used for docking analysis were obtained from RCSB PDB. Among the immune-related proteins expressed in humans (or monkeys) and mice, 41 protein structures related to innate immunity and macrophage activation were selected by referring to Innated. After excluding 16 inappropriate structures, 25 receptor protein structures were introduced for reverse docking. The reverse docking analysis was performed by using AutoDock Vina in AMDOCK. The selected PDB protein structures were converted to PQR files in AMBER force field in advance. 6 galactose oligosaccharides were prepared as putative ligands in ChemDraw (PerkinElmer) and subjected to energy minimization before being introduced into docking. The electrostatic potential of the protein surface was calculated by using APBS (Adaptive Poisson Boltzmann Solver). The visual representation of proteins and other molecules was depicted by using PyMOL. The mouse TLR4/MD2 structure 3VQ2, which was shown to have the lowest binding energy in the docking analysis, was introduced into molecular dynamics (MD) simulations by using NAMD. The ligand-free, LPSbound and Gal9-docked 3VQ2 PDB structures were converted to PSF files by applying the CHARMM force field and then applying solvation and ionization by the VMD plugin. The simulations were performed at 300 K for 100 ns. Langevin dynamics was applied throughout the simulation 16. The timestep parameter was set to 2 fs, and coordinates were recorded every 1,000 steps. All trajectory analyses were performed by using the VMD plugin.

### 1.9. Surface Plasmon Resonance (SPR) for Evaluation of Target Protein Binding Affinity

Surface plasmon resonance (SPR) analysis was performed on Biacore T200 (GE Healthcare) to investigate the interaction between recombinant human TLR4 (hTLR4, Sino Biological) and ligands, the carbohydrate immunomodulatory anti-tumor adjuvant constructs or LPS. hTLR4 was purified by centrifugal filtration using running buffer (PBS supplemented with 0.005% Tween-20). hTLR was immobilized by using an NTA sensor chip (GE Healthcare). Before each run, the NTA sensor chip was loaded with Ni²⁺ ions by treating with 500 mM nickel chloride at 10 L/min for 1 minute. hTLR was then immobilized on the surface chip at a density of 350 RU. Sensorgrams of the binding of the carbohydrate immunomodulatory anti-tumor adjuvant constructs or LPS to hTLR were obtained through continuous analyte increase at a flow rate of 30 L/min. The association and dissociation periods were 120 seconds and 200 seconds, respectively. After the dissociation step was completed, 0.35 M EDTA solution was injected for surface regeneration. The BIAevaluation program was used for data analysis. The association (kon) and dissociation (koff) rate constants were determined by assuming the Langmuir binding model and multivalent stoichiometry. The dissociation constant (KD) was calculated by comparing the kinetic rate constants as KD=koff/kon. In order to confirm the formation of the actual complex, the complex of recombinant TLR4 and the carbohydrate immunomodulatory anti-cancer adjuvant construct was detected by size exclusion chromatography. First of all, 100 μg of recombinant TLR4 (rTLR4) standard protein was redistributed in 100 μL of sterile water and purified by using a PD-10 desalting column to remove the preservative. The carbohydrate immunomodulatory anti-cancer adjuvant construct was sonicated for 30 minutes and reacted with purified rTLR4 at 37° C. for 3 hours. For complexation of the carbohydrate immunomodulatory anti-tumor adjuvant construct with 17 rTLR4, the molar ratio was maintained at 5:1 (carbohydrate to protein). The carbohydrate immunomodulatory anti-tumor adjuvant construct-TLR4 complex was analyzed by using a 1260 HPLC-DAD system (Agilent) with an AdvanceBio SEC column (4.6×300 mm, 2.7 m, 300 Å, Agilent). The peaks of unbound protein or complex were detected at a wavelength of 280 nm. The molecular masses of the proteins and complexes were estimated by using standard curves obtained from the reference proteins thyroglobulin dimer (1,340 kDa), thyroglobulin (670 kDa), immunoglobulin G (150 kDa), bovine serum albumin (66 kDa), ovalbumin (44 kDa) and lysozyme (14.3 kDa).

### 1.10. Colocalization Analysis Using Fluorescence Cell Imaging

The carbohydrate immunomodulatory anti-cancer adjuvant construct (1 mg) was reconstituted in 1 mL of activation buffer (0.1 M MES, 0.5 M NaCl, pH 6.0), and then, 0.4 mg of EDC (Sigma) and 1.1 mg of Sulfo-NHS (Thermo Scientific) were completely mixed with the carbohydrate immunomodulatory anti-cancer adjuvant construct solution and the reaction components, and the mixture was incubated at room temperature for 15 minutes. Next, Cyanine-3 (Cy3) amine (Abcam) was added to the mixture and reacted at room temperature for 20 hours. After incubation, the reaction was stopped by adding excess hydroxylamine. Cy3-conjugated carbohydrate immunomodulatory anti-cancer adjuvant construct (Cy3-carbohydrate immunomodulatory anti-cancer adjuvant construct) was purified by using a HiPrep 26/10 Ddsalting column (Cytiva) on an AKTA Purity System (Cytiva). The purified Cy3-carbohydrate immunomodulatory anti-cancer adjuvant construct was analyzed by using a 1260 HPLC system (Agilent) which was equipped with a Series 200a fluorescence detector (PerkinElmer). For colocalization analysis, bone marrow-derived macrophages (BMDMs) (1×10⁵ cells) were seeded onto 35 mm confocal dishes and cultured overnight, followed by brief exposure to the Cy3-carbohydrate immunomodulatory anti-cancer adjuvant construct (10 μg/mL) for 3 minutes at 37° C. Afterwards, cells were then washed with cold PBS to remove the unbound Cy3-carbohydrate immunomodulatory anti-cancer adjuvant construct and fixed with 4% paraformaldehyde. Fc receptors were blocked with anti-CD16/CD32 antibodies, and FITC-conjugated anti-mTLR4 antibodies were used. Afterwards, the cells were then washed and mounted on coverslips. Fluorescence imaging was performed by using a TCS SP8 confocal microscope (Leica). Acquired images were analyzed by using ImageJ on the Fiji platform, and image-based colocalization was assessed by using the Coloc2 and JaCoP plugins for ImageJ.

### 1.11. Preparation of Murine Bone Marrow-Derived Macrophages (BMDM)

BMDMs were obtained from the bone marrow of femurs and tibias of C57BL/6 mice. Primary cells were isolated from the bone marrow of 6 to 8-week-old C57BL/6 mice (18-20 g) and then treated with red blood cell lysis buffer (eBioscience). After filtering through a 100 m cell strainer, the primary cell suspension was maintained in Dulbecco's modified Eagle's medium (DMEM, Gibco) containing 10% FBS, 1% penicillin-streptomycin and 10 ng/mL recombinant murine macrophage colony-stimulating factor (M-CSF) for 6 days. Half of the bone marrow culture medium was replaced with fresh DMEM containing M-CSF on day 4. On day 7, the culture medium was replaced with medium without M-CSF, and the harvested BMDMs were directly introduced into the subsequent in vitro experiments. All mice were housed in individual cages and were provided with food and water ad libitum throughout the study. Mice were observed for at least 2 weeks prior to the start of the study to determine any abnormalities. All animal experiments were approved in advance by the Institutional Animal Care and Use Committee (IUAUC) (SNU-210901-2) and were performed in strict compliance with the IUAUC guidelines.

### 1.12. Immunoreceptor Signaling Assay

Cells were lysed in M2 buffer, and the composition of M2 buffer was as follows: 20 mM Tris at pH 7, 0.5% NP-40, 250 mM NaCl, 3 mM EDTA, 3 mM EGTA, 2 mM DTT, 0.5 mM PMSF, 20 mM P-glycerol phosphate, 1 mM sodium vanadate and 1 mg/mL leupeptin. Cell extracts obtained therefrom were resolved by using SDS-PAGE, analyzed using immunoblotting, and analyzed using chemoillumiaescence (Pierce™ ECL Western Blotting Substrate, 32,106). The activation of TLR signaling was also assessed by measuring the amount of secreted embryonic alkaline phosphatase (SEAP) produced by HEK-Blue mTLR4 HEK293 reporter cells (InvivoGen). LPS was used as a reference ligand (0.05 μg/mL) to assess TLR4 stimulation. As test ligands, LPS and anionic carbohydrates including the carbohydrate immunomodulatory anti-tumor adjuvant construct, 50 kDa hyaluronan (Creative PEGworks) and 250 kDa carboxymethylcellulose (Sigma) were incubated with SEAP reporter cells for 24 hours. Secreted SEAP levels were quantified by incubating the cell medium with Quanti-Blue solution (InvivoGen) for 6 hours and measured at a wavelength of 650 nm. For TLR4 inhibition assay, RAW264.7 cells were treated with a selective TLR4 signaling inhibitor, LPS-RS (1 μg/mL), LPS-RS ultra (1 μg/mL), L48H37 (10 μM) and Naloxone (2 mM). NO release induced by the carbohydrate immunomodulatory anti-cancer adjuvant construct (1 μg/mL) was tested by using the Griess assay to assess macrophage stimulating activity.

### 1.13. Cytotoxicity Assay

In order to assess cytotoxicity, cell viability was determined based on the lactate dehydrogenase (LDH) leakage test, and the results were quantified by using the CytoTox96® Non-Radioactive Cytotoxicity Assay Kit (Promega, G1780). LDH absorbance was measured at a wavelength of 490 nm. The absorbance signal was measured by using a POLARstar OPTIMA Multidetection microplate reader. For Annexin V testing using flow cytometry, cell suspensions were stained on ice for 20 minutes in the dark with various combinations of fluorochrome-conjugated antibodies, and the FITC Annexin V Apoptosis Detection kit (BD Biosciences, 556,547) was used.

RNA sequencing (RNA-seq) total RNA was isolated by using Trizol reagent (Invitrogen). The purity of RNA was assessed by using an Agilent 2100 Bioanalyzer with an RNA 6000 Nano Chip (Agilent Technologies) and quantified by using an ND-2000 spectrophotometer (Thermo Scientific). For control and test RNA, library construction was performed by using the QuantSeq 3′ mRNASeq Library Prep Kit (Lexogen) according to the protocol. Briefly, 500 ng of total RNA was prepared, and oligo-dT primers including an Illumina-compatible sequence at the 5′ end were hybridized to the RNA and reverse transcribed. After digestion of the RNA template, second-strand synthesis was initiated by using a random primer including an Illumina-compatible linker sequence at the 5′ end. The double-stranded library was purified by using magnetic beads to remove all reaction components. The library was amplified to add the complete adapter sequence required for cluster generation. The final library was purified from the PCR components. High-throughput sequencing was performed by using NextSeq 500 (Illumina) with single-end 75 sequencing. QuantSeq 3 mRNA-Seq reads were aligned by using Bowtie2. Bowtie2 indices were generated from representative transcript sequences for alignment to genome assembly sequences or genome and transcriptome. Alignment files were used for transcriptome assembly, abundance estimation and differential expression detection of genes. Differentially expressed genes (DEGs) were determined based on the number of unique multiple alignments using Bedtools' range. RC (read count) data were processed by using Bioconductor using EdgeR in R with the TMM+CPM normalization method. Gene classification was based on searches performed using DAVID and Medline databases.

### 1.14. Expression Gene Set Analysis and Meta-Comparison

Gene set enrichment analysis (GSEA) was performed by using GSEA 4.2.3 software provided by the Broad Institute, UC San Diego. A total of 186 gene sets from the canonical pathways (CPs) of KEGG selected from MsigDB were used for GSEA. Only one gene set was excluded by the size filter (min=10, max=500). GO (Gene Ontology) enrichment analysis was performed by using ShinyGO 0.76 with differentially expressed genes identified using RNA-seq. The FDR cutoff was set at 0.05. Transcriptomic meta-comparative analysis of immune-related gene signatures was performed by using two gene sets (GSE155801/GSE163347) published in the GEO database. The gene expression levels of each gene set were compared based on the TMM-normalized values.

### 1.15. Immunosuppressive Macrophage Re-Education Assay

For the immunosuppressive macrophage model established using TL-4 ((M(IL-4)), BMDMs were prepared in 12-well plates (1×10⁶ cells/well) and then treated with 40 ng/mL mouse IL-4 in DMEM for 24 hours. For the tumor immunosuppressive macrophage model (M(tumor)), MC38 cells were seeded (5×10⁵ cells/well) on the apical side of 12-well Transwell® plates with 3.0 m pores (Corning) and cultured to confluence. Afterwards, BMDMs were then placed on the basolateral side of the Transwell (1×10⁶ cells/well) without changing the culture medium for 2 days and stimulated for 24 hours to obtain the immunosuppressive macrophage model. In the in vitro environment, two strategies were used to differentiate macrophages into immunosuppressive cells. For cell phenotypic analysis, cultured cells were harvested, and flow cytometric analysis was performed. Cells were detached, washed and collected by centrifugation in cold flow cytometric staining buffer. Next, 0.5 μg/mL CD16/CD32 antibodies were added to the cells and incubated on ice for 30 minutes to block Fc receptors. For surface staining, primary antibodies conjugated with fluorescent dyes were directly added to the cells and then incubated on ice for 1 hour. For intracellular staining, cells were fixed and permeabilized by using the Foxp3/transcription factor staining buffer set according to the manufacturer's protocol. Intracellular antibodies were added to the cells and incubated for 30 minutes at room temperature. The prepared cells were analyzed by using an LSRFortessa™ flow cytometer (BD Bioscience). Data analysis was performed by using FlowJo 10.4 software (Beckton Dickinson). For RT-qPCR, total RNA was extracted from BMDMs by using the AccuPrep® Universal RNA Extraction Kit (Bioneer). RT-qPCR was performed 3 times by using the AccuPower® GreenStar™ RT-qPCR Master Mix (Bioneer). The relative expression of genes (Il1b, Nos2, Il6, Tnfa, Arg1, Vegfa, Tgfb1 and Il10) was measured by using a QuantStudio™ 6 Flex Real-time PCR system (Applied Biosystems) and normalized to the expression of Gapdh. In order to assess cytokine production in response to stimuli for in vitro cytokine and lipid mediator quantification, supernatants from BMDM culture media were collected and analyzed by using ELISA kits (Invitrogen) according to the manufacturer's protocol. For lipid mediator analysis, lipid mediators in cell culture media were extracted by using solid phase extraction (SPE). Extraction recoveries were assessed by using an internal standard (d4-PGE2; Cayman Chemical). After extraction using an Oasis HLB SPE column (Waters), the solvent was evaporated under a stream of nitrogen gas, and the samples were resuspended in a methanol/water solution (50:50). Samples were then analyzed by using high-performance liquid chromatography with a 6460 triple-quadruple mass spectrometer (Agilent) in negative ion mode. Identification was performed by matching retention times with authentic standards, MRM transitions and fragment ions.

### 1.16. Anti-Cancer Efficacy Experiments in Mouse Tumor Growth Models

In the MC38 colon adenocarcinoma subcutaneous model, MC38 (Kerafest, Boston, MA) or MC38-Luc-RFP (Alstem, Richmond, CA) cells suspended in 50 μL of PBS were injected subcutaneously into C57BL/6 or BALB/c-nude mice. When tumors reached a size of 25 mm² (100 mm³), mice were randomly divided and assigned to cohort groups. In order to deplete intratumoral macrophages in C57BL/6 mice, clodronate liposomes (5 mg/mL in PBS, 50 μL) were injected intratumorally 1 day before the injection of the carbohydrate immunomodulatory anti-tumor adjuvant construct or PBS, with additional injections given 7 and 14 days after the initiation of treatment. The successful depletion of macrophages was confirmed by lysing tumor tissue and measuring the number of CD11b+F4/80+ macrophages using flow cytometry. Animals were administered intratumorally the carbohydrate immunomodulatory anti-cancer adjuvant construct (2.5 mg/kg) in 50 μL PBS or just 50 μL PBS every other day. In the B16-F10 melanoma subcutaneous model, B16-F10 cells (Alstems) suspended in 50 μL of PBS were injected subcutaneously into C57BL/6 mice. For combination studies with anti-PD-1 (αPD-1), after the tumors reached a size of 25 mm² (100 mm³), the mice were randomly divided into 4 different groups (control, carbohydrate immunomodulatory anti-cancer adjuvant construct-treated group, αPD-1-treated group, αPD-1+carbohydrate immunomodulatory anti-cancer adjuvant construct-treated group). For αPD-1 pretreatment, RMP1-14 anti-PD-1 clone (BioXcell) was administered intraperitoneally at a dose of 200 μg daily prior to the initiation of carbohydrate immunomodulatory anti-tumor adjuvant construct or PBS treatment (day 0). The carbohydrate immunomodulatory anti-tumor adjuvant construct (2.5 mg/kg) in 50 μL PBS or 50 μL PBS alone was administered intratumorally every other day. Tumor volume (V) was measured every other day by using calipers (V=0.50×short axis diameter 2×long axis diameter), and results were reported after normalization to baseline volume. Survival follow-up was performed for a total of 28 days. Mice were euthanized at the end of the experiment, and tumors were dissected, weighed and photographed. All mice were housed individually and had free access to food and water throughout the study. Mice were observed for at least 2 weeks prior to the start of the study to check for evidence of preexisting abnormalities. All animal handling was performed strictly in accordance with IUAUC guidelines. In order to phenotypically characterize the immunological status of tumors, mice were euthanized by CO₂ inhalation at the end of the experiment, and tumors were harvested. Tumor tissue was cut into small pieces, minced and further incubated in RPMI supplemented with 0.2 mg/mL collagenase I (Merck) and 50 U/mL DNase I (Roche) at 37° C. for 30 minutes. Afterwards, cells were then passed through a 70 m cell strainer before lysing red blood cells with RBC lysis buffer (eBioscience) on ice for 3 minutes. For the isolation of tumor-infiltrating leukocytes (TILs), cells were resuspended in RPMI medium, and mononuclear cells were isolated by using Ficoll-Paque density gradient centrifugation. Briefly, 30 mL of cell suspension was centrifuged at 1,025 g for 20 minutes with 10 mL of Ficoll-Paque PREMIUM (Cytiva). Next, cells were then harvested from the interface between the medium and Ficoll-Paque. Cells were washed twice with flow cytometry staining buffer (eBioscience) and incubated with 0.5 μg/mL CD16/CD32 antibodies (Biocarbohydrate immunomodulatory anti-tumor adjuvant construct) for 30 minutes for Fc blocking. Afterwards, the cell surface was then stained with antibodies labeled with fluorescent dyes. Next, cells were then labeled with Fixable Viability Dye eFluor™ 455UV (eBioscience) to determine viability. For intracellular staining, cells were fixed and permeabilized by using the Foxp3/Transcription Factor Staining Buffer Set (eBioscience) according to the manufacturer's protocol. Intracellular antibodies were added to the cells and incubated for 30 minutes at room temperature. The prepared cells were analyzed by using an LSRFortessa™ flow cytometer (BD Bioscience). Data analysis was performed by using FlowJo 10.4 software (Beckton Dickinson). For the immunohistochemical analysis of tumors, after additional harvest, tumor tissues were rapidly preserved by rapid freezing in isopentane cooled to liquid nitrogen (−150° C.). Frozen tissues were sectioned at 8 m thickness on slides by using a CM3050S cryostat (Leica) and then fixed in 4% paraformaldehyde. Slides were washed and blocked with 5% bovine serum albumin and anti-CD16/CD32 antibodies. For immunofluorescence staining, tumor sections were incubated with dye-conjugated primary antibodies to Ly6C (Biocarbohydrate Immunomodulatory Anti-tumor Adjuvant Structureend) and CD206 (Biocarbohydrate Immunomodulatory Anti-tumor Adjuvant Structureend) for 1 hour at room temperature and washed 3 times with PBS. Cell nuclei were counterstained with DAPI. Stained sections were analyzed by using a TCS SP8 confocal microscope (Leica). Image processing, analysis and quantification were performed by using LAS X software (Leica).

### 1.17. Near-Infrared Fluorescence (NIRF) Imaging for Confirmation of In Vivo Distribution

The NIRF probe targeting surface CD206 was prepared by conjugating Cy7 fluorescent dye to 2-amino-2-deoxymannose. Briefly, 1 mol of Cy7 NHS ester (Lumiprobe) was conjugated with 10 mol of mannosamine hydrochloride in 0.05 M sodium borate buffer (pH 8.5). After incubation at 4° C. for 2 hours in the dark, the conjugated Cy7-conjugated mannose (Man-Cy7) was purified by using a preparative C18 HPLC column (Phenomenex) on a 321 preparative-LC system (Gilson). The purified Man-Cy7 NIRF probe was identified by using 1260 HPLC with a 6460 triple-quadruple mass spectrometer (Agilent). For NIRF imaging, mice were anesthetized with 2% isoflurane and injected intraperitoneally with Man-Cy7 (200 μL, 5 nmol). After 4 hours, in vivo imaging was performed by using an IVIS Spectrum system (PerkinElmer), where Cy7 fluorescence was measured at excitation and emission wavelengths of 710 nm and 800 nm, respectively. In vivo image analysis was performed by using Living Image 4.5 software (PerkinElmer).

### 1.18. Statistical Analysis

Statistical analysis data were expressed as mean with error bars representing standard deviation (S.D.) or standard error of the mean (S.E.M.). Experimental data were analyzed with Graphpad 9 and R version 4.2.

### Example 2. Experimental Results

### 2.1. Design of Carbohydrate Immunomodulatory Anti-Cancer Adjuvant Construct Through Verification of Immune-Active Molecular Moiety of Raw PUP

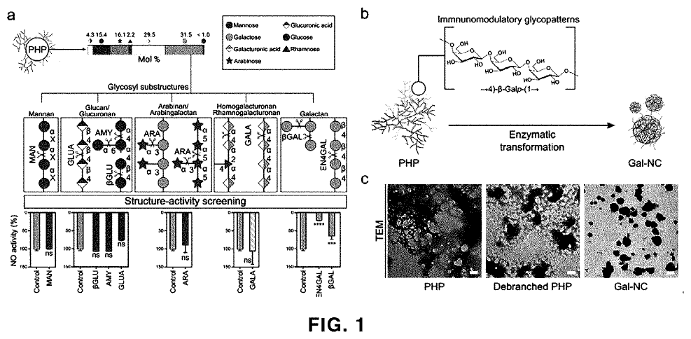

Standard pectin heteropolysaccharide (PUP) is a natural carbohydrate polymer with an immunostimulatory molecular pattern that is recognized by innate immune receptors. In order to design multimeric carbohydrate heteromers from PUP, the active moiety of PUP had to be distinguished. Therefore, a panel of site-specific carbohydrate-active enzymes was constructed based on the substructure of PUP to investigate the structure-activity relationship. The change in activity after side chain cleavage by a specific enzyme can indicate the relationship between bioactivity and the structure cleaved by this enzyme. A significant decrease in activity was observed after treatment with enzymes that selectively remove galactan branches. In particular, the treatment of 1,4-β-endogalactanase reduced the activity by 78% compared to the control. In contrast, no significant decrease in activity was observed in the selective cleavage of most other structures such as mannan, arabinan and glucan. These results indicate that the galactan structure is the active moiety of PUP (FIG. 1a). Based on these results, the carbohydrate immunomodulatory anti-cancer adjuvant construct were constructed based on the PUP structure to improve molecular activity, stability and self-assembly properties. In addition, the inactive glycosyl branches of PUP were intentionally removed, and the active macromolecular scaffold was obtained through the optimization of enzyme production, followed by performing preparative size exclusion chromatography (Prep-SEC) separation (FIGS. 1b and c).

### 2.2. Preparation and Physical Properties of Carbohydrate Immunomodulatory Anti-Cancer Adjuvant Constructs

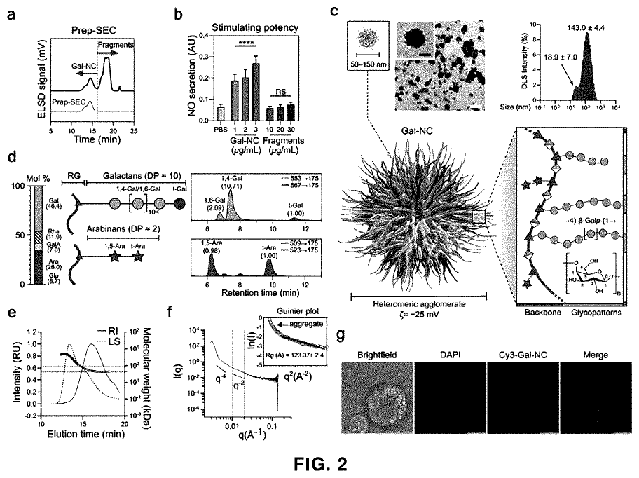

The carbohydrate immunomodulatory anti-cancer adjuvant construct is a modified macromolecular carbohydrate including galactan repeat structures (FIG. 2a). After Prep-SEC, the macromolecular scaffold including galactan branches was separated from the low-molecular-weight fraction. The integrity of the active portion was confirmed in that the macrophage stimulating activity remained only in the high-molecular-weight fraction. In addition, the absence of LPS contamination was confirmed (FIG. 2b). The scaffold spontaneously aggregated at physiological pH (˜7) via intermolecular electrostatic forces, thereby exhibiting a weakly anionic property with a zeta potential of about −25 mV, and dynamic light scattering (DLS) analysis revealed that the scaffold had a hydrodynamic diameter of about 140 nm (FIG. 2c). The branched structure of the carbohydrate immunomodulatory anti-cancer adjuvant construct was further investigated. The galactan/arabinan side chains attached to the RG backbone of the carbohydrate immunomodulatory anti-cancer adjuvant construct were further identified by using ¹H-¹³C HSQC NMR. The average degree of polymerization (DP) of each side chain was calculated by using the composition ratio of internal and terminal sugars. The average DP of galactan residues was about 10, whereas the average DP of arabinan residues was about 2 (FIG. 2d). The estimated molecular mass of the scaffold molecules was about 203 kDa with aggregates larger than 1,000 kDa as measured by using light scattering (FIG. 2e). By using small-angle X-ray scattering (SAXS) analysis of the diluted construct solution, the radius of gyration (Rg) of the chain was calculated to be approximately 123.37 Å in the Guinier (qRg≤1.3) region, but the low q region indicates the presence of larger aggregates (FIG. 2f). The repeated galactan branches form an intermolecularly entangled brush-like polymer, thereby imparting supramolecular properties that are different from the parent PUP. Fluorescence microscopy experiments confirmed that the construct molecules had the property of binding to and aggregating on the surface of macrophages (FIG. 2g).

### 2.3. Binding Simulation for TLR4 and Experimental Confirmation of Complex Formation

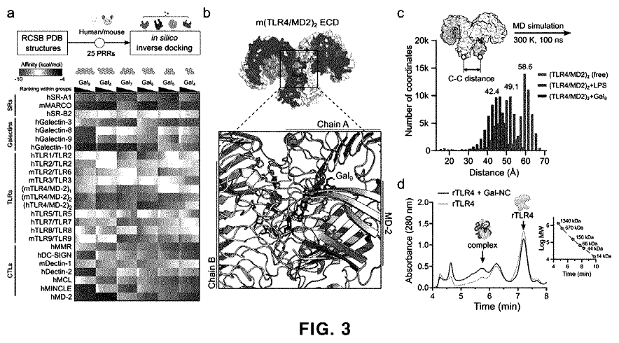

The molecular recognition mechanism of the carbohydrate immunomodulatory anti-cancer adjuvant construct for the TLR4 receptor was investigated. To this end, the binding energy was estimated through in silico reverse docking analysis from the simplified interaction between the predesigned galactose oligosaccharide (GOS) and the receptor protein structure. A total of 25 receptor structures were selected from the Protein Data Bank (PDB) (FIG. 3a). The affinities of the top 10 docking poses were calculated by using AutoDock Vina and AMDOCK to mutually dock 6 types of GOSs to the selected PDB structures. The dimeric TLR4 structure formed the most stable docked complex with GOSs of all complexes. In this complex, nona-galactan (Gal9) was located between the two horseshoe-shaped leucine-rich regions (LRRs) of the TLR4/MD-2 complex (3VQ2) (FIG. 3b). The best-docked poses of Gal9 and 3VQ2 were introduced into molecular dynamics simulations using NAMD. During the molecular dynamics simulation for 100 ns, the distance between the C-termini of the two main chains of TLRs (C-C distance) was the shortest when Gal9 was the ligand (FIG. 3c). The C-C distance is closely related to the dynamic stability of TLR4 dimerization, because the C-terminus of the 3VQ2 structure is the site where the extracellular domain of TLR4 connects with the transmembrane domain. In order to verify this experimentally, the carbohydrate immunomodulatory anti-cancer adjuvant construct was mixed with recombinant hTLR4 and incubated in phosphate-buffered saline (PBS) at 37° C. for 5 hours to investigate the binding properties of the carbohydrate immunomodulatory anti-cancer adjuvant construct to hTLR4 in a dispersed physiological solution. After incubation as a 1:1 weight mixture for 5 hours under physiological conditions (37° C. in PBS), a broad peak indicating approximately 360 kDa appeared at 5.7 minutes. The SEC chromatogram using molecular weight standards showed the formation of a high molecular weight protein-carbohydrate complex of approximately 360 kDa (FIG. 3d).

### 2.4. Analysis of the Multivalent Binding Pattern for TLR4

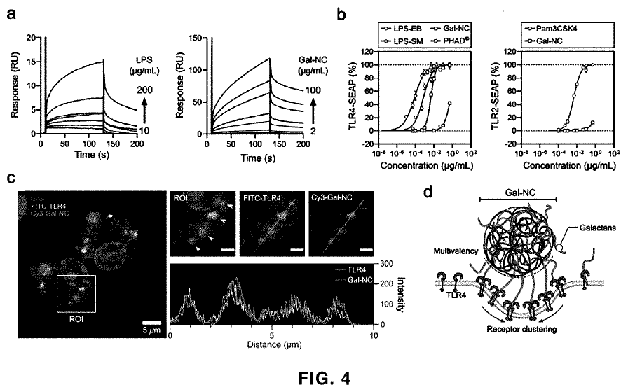

The recombinant human TLR extracellular domain (hTLR4-ECD) protein was immobilized on a chip surface through the interaction between Ni-NTA and the C-terminal His-tag to obtain a vertical arrangement mimicking the orientation of hTLR4 on the cell surface, and the binding affinity was determined for the size-purified 1,4-β-galactan (FIG. 4a). In addition, when it was treated with various binding inhibitors, it was confirmed that the construct induced TLR4-specific activation (FIG. 4b). When the carbohydrate immunomodulatory anti-cancer adjuvant construct with galactan branches was recognized by macrophages, a set of receptors accumulated at the carbohydrate immunomodulatory anti-cancer adjuvant construct binding site on the cell membrane, resulting in spatial receptor clustering, as confirmed by colocalization analysis under fluorescence microscopy (FIG. 4c). Overall, the results suggest that the carbohydrate immunomodulatory anti-cancer adjuvant construct including the galactose oligosaccharide structure binds to TLR4 in a manner different from that of LPS, which is a well-studied ligand of TLR4 (FIG. 4d).

### 2.5. Analysis of Immune-Stimulatory TLR4 Signaling Pathway Activation in Macrophages

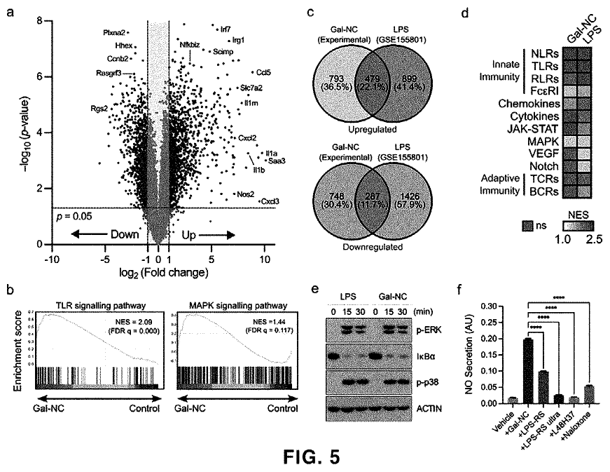

Activation of the TLR4 signaling pathway is associated with the transcriptional regulation of numerous gene expressions that induce inflammatory responses. Therefore, it was investigated whether treatment with the construct altered the gene expression pattern of BMDMs. The RNA sequencing data set obtained from macrophages treated with the carbohydrate immunomodulatory anti-cancer adjuvant construct was analyzed, and it was confirmed that genes associated with immune stimulation and early inflammatory responses, such as Il1a, Ccl5 and Cxcl2, were upregulated after treatment (FIG. 5a). As a result of the gene expression enrichment analysis of differentially expressed genes (DEGs), it was confirmed that these DEGs were closely related to TLR signaling and MAPK signaling pathways (FIG. 5b). Next, immune-related DEGs were classified for meta-comparison with the published Gene Expression Omnibus dataset (GES155801) that shows transcriptome changes in LPS-stimulated BMDMs. 479 up-regulated genes and 287 down-regulated genes were commonly identified in the DEGs of both groups (FIG. 5c). It as confirmed that the gene cluster changes induced by the construct were more specific to the innate immune signaling pathway than those induced by LPS (FIG. 5d). This pathway showed TLR4 downstream signaling such as IκBα degradation and ERK/p38 phosphorylation (FIG. 5e). Next, it was investigated whether the carbohydrate immunomodulatory anti-cancer adjuvant construct could induce NO secretion in a TLR4-specific manner. In murine TLR4 reporter HEK293 cells, the carbohydrate immunomodulatory anti-cancer adjuvant construct-mediated NO secretion was strongly reduced by various TLR4 inhibitors, indicating that the construct-induced macrophage activation was dependent on TLR4 stimulation (FIG. 5f).

### 2.6. Analysis of Re-Education of Immunosuppressive M2 Macrophages into Immunoactive M1 Macrophages Through Adjuvant Treatment

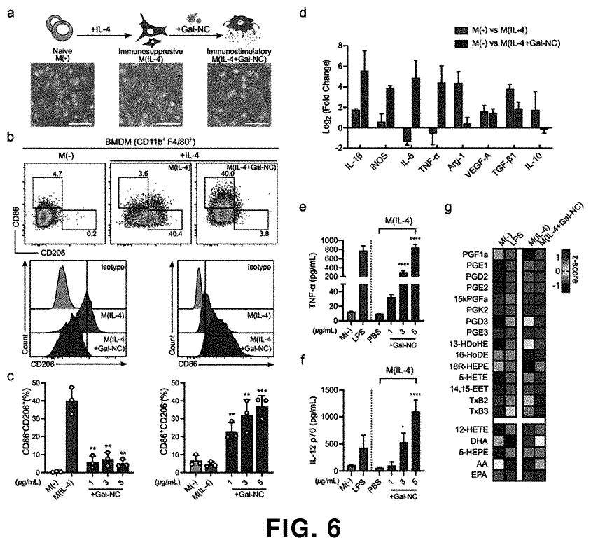

M2 immunosuppressive macrophages by Th2 cytokines (IL-4 and IL-13) can be functionally reprogrammed into an M1 immunostimulatory phenotype when exposed to LPS or other innate stimuli. In order to investigate the possibility that the carbohydrate immunomodulatory anti-cancer adjuvant construct could polarize macrophages, M2-like macrophage models were obtained through IL-4 treatment or co-culture with MC38 colon adenocarcinoma cells (FIG. 6a). In order to investigate whether the carbohydrate immunomodulatory anti-cancer adjuvant construct induces phenotypic changes in M2-polarized macrophages under the conditions mentioned above, the treatment of the carbohydrate immunomodulatory anti-cancer adjuvant construct was performed. The treatment of the carbohydrate immunomodulatory anti-cancer adjuvant construct induced morphological changes and expanded the cytoplasmic area of immunostimulatory macrophages with phagosomes in both M(IL-4) and M(tumor) models. As a result of flow cytometric analysis of macrophage surface markers, it showed that treatment with the carbohydrate immunomodulatory anti-cancer adjuvant construct significantly reduced the expression of immunosuppressive CD206+ and increased the expression of immunostimulatory CD86+ (FIG. 6b). IL-4-induced M2-polarized macrophages showed a dramatic decrease after treatment with different concentrations of the carbohydrate immunomodulatory anti-cancer adjuvant construct, whereas the CD86+CD206− population increased in proportion to the carbohydrate immunomodulatory anti-cancer adjuvant construct (FIG. 6c). In the ELISA results for specific gene expression analysis, the expression levels of 4 immunostimulatory genes encoding IL-1b, iNOS, TL-6 and TNF-α were significantly increased, whereas the expression levels of 4 representative immunosuppressive genes encoding Arg-1, VEGF-A, TGF-β1 and IL-10 were significantly increased and down-regulated in response to the construct treatment (FIG. 6d). For changes in immune-related gene expression, a dose-dependent increase at the levels of immunostimulatory cytokines TNFα and IL-12 p70 was observed (FIGS. 6e and f). In addition, according to lipid mediator analysis, it showed an overall increase in other inflammatory lipid mediators in the construct-treated M(IL-4) model (FIG. 6g). These results demonstrate that the carbohydrate immunomodulatory anti-cancer adjuvant construct reprogrammed M2-polarized macrophages to an M1-like phenotype in a TLR4-dependent manner.

### 2.7. Tumor Growth Reduction Efficacy Through Tumor Macrophage Re-Education in In Vivo Model

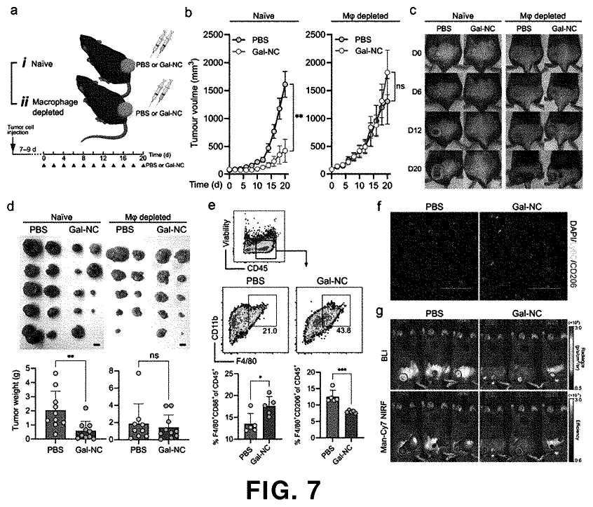

Next, it was investigated whether the effect of the carbohydrate immunomodulatory anti-cancer adjuvant construct on inducing TAMs to an activated M1-like phenotype suggests potential anti-tumor therapeutic efficacy in tumor-bearing mice. Two models were established with MC38 murine colon cancer cells in both normal C57BL/6 mice (naïve) and macrophage-depleted C57BL/6 mice (FIG. 7a). When tumor volumes reached 0.3 to 0.5 cm, PBS (control) or the carbohydrate immunomodulatory anti-tumor adjuvant construct (2.5 mg/kg⁻¹) was injected intratumorally every other day and followed for up to 20 days. The construct inhibited the growth of MC38 tumors in C57BL/6 mice (FIG. 7b to d). However, more interestingly, in the second tumor model, where macrophages were depleted with clodronate liposomes prior to the injection of PBS or the carbohydrate immunomodulatory anti-tumor adjuvant construct, construct treatment did not inhibit tumor growth, suggesting a pivotal role for intratumoral macrophages in the tumor microenvironment. After tumor size tracking, tumor tissues were harvested and analyzed for the TAMs population by FACS and immunofluorescence staining. The number of TAM subset (CD11b+F4/80+) of total leukocytes (CD45+) in the tissues was significantly increased from 21.0% to 43.8% in tumors treated with the carbohydrate immunomodulatory anti-cancer adjuvant construct compared with the control group (FIG. 7e, top). The proportion of immunostimulatory macrophages (F4/80+CD86+) increased by an average of 23% in the construct-treated group, while the proportion of immunosuppressive macrophages (F4/80+CD206+) decreased by an average of 37% (FIG. 7e, bottom). Tissue immunofluorescence staining showed that construct treatment decreased CD206 expression but increased Ly6C expression compared with the control group (FIG. 7f). In order to further determine the macrophage-dependent anti-tumor efficacy of the carbohydrate immunomodulatory anti-tumor adjuvant construct, a fluorescent probe targeting CD206 was designed as previously described and injected intraperitoneally into MC38-Luc-RFP tumors on day 10 of treatment. Consistent with the decrease in tumor bioluminescence after construct treatment, CD206 fluorescence decreased at the tumor site (FIG. 7g). Taken together, the results suggest that the construct induces the repolarization of immunosuppressive TAMs toward anti-tumor dominance, and the tumor suppression efficacy of the carbohydrate immunomodulatory anti-tumor adjuvant construct may be attributed to macrophage stimulation activity.

### 2.8. Synergistic Effect of Anti-Tumor Activity in Combination with Carbohydrate Immunomodulatory Anti-Tumor Adjuvant Construct in Anti-PD-1 Immunotherapy

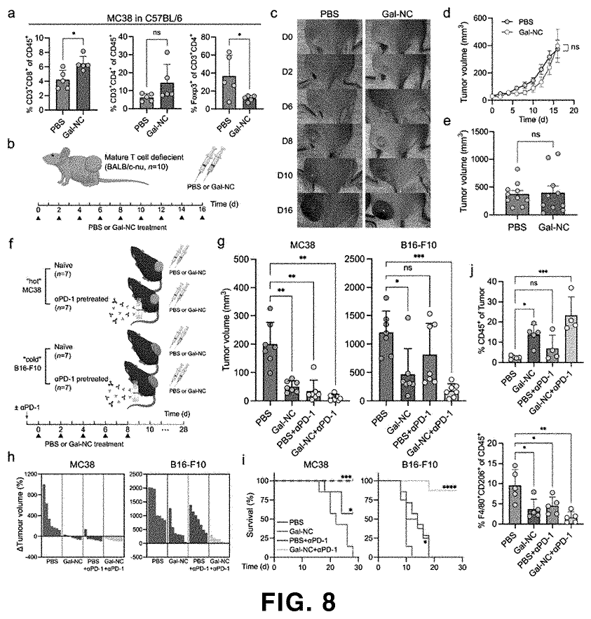

As a result of analysis of the distribution of T cell subsets in tumors, it showed that the composition of CD8+ cytotoxic T lymphocytes (CTLs), which is the most prominent killer factor in tumor and cancer immunotherapy, was significantly increased in the construct-treated group. Although construct treatment did not increase the total number of CD4+ helper T cells in the tumor, the number of regulatory T cells (CD4+Foxp3+) that suppress CTLs was significantly reduced to less than 15% of the total helper T cell number in the construct-treated group (FIG. 8a). This intratumoral T cell profile strongly supports that construct treatment can alter the TME to favor anti-tumor T immunity. Construct treatment did not show any anti-tumor effect in T-deficient mice when followed up, suggesting that anti-tumor T immunity is required for construct-mediated tumor suppression (FIG. 8b-e). Next, the potential of the construct as an adjuvant to enhance the therapeutic efficacy of immune checkpoint inhibitors (ICBs) in combination was analyzed. First of all, B16-F10 melanoma (hypoimmune-responsive tumor) and MC38 colon cancer (immunoreactive tumor) models, which are highly and lowly responsive to anti-PD-1 (PD-1) mAb immunotherapy, respectively, were established (FIG. 8f). High responses were observed with monotherapy with the construct or αPD-1 mAb, while combination therapy resulted in effective tumor regression and improved survival. The B16-F10 model showed a typical cold tumor phenotype with lower responses to all treatments than the MC38 model, as expected. Interestingly, in a low-responsive tumor model, monotherapy with the construct showed better anti-tumor efficacy than monotherapy with αPD-1 mAb, and significantly improved anti-tumor efficacy when combined with αPD-1 mAb (FIG. 8g to i). When the proportion of immune cells in the tumor was analyzed, all construct-treated groups showed an average greater than 10% increase in total tumor leukocyte count, while the construct/anti-PD-1 mAb combination group had an average leukocyte proportion of 20% or more. In addition, the proportion of immunosuppressive macrophages (F4/80+CD206+) was significantly reduced, indicating that the carbohydrate immunomodulatory anti-cancer adjuvant construct can mainly increase the proportion of immune cells that are beneficial to anti-tumor immunity in B16-F10 tumors (FIG. 8j).

In summary, the present invention identified the active moiety of pectin heteropolysaccharide (PUP) and developed a carbohydrate complex called a carbohydrate immunomodulatory anti-cancer adjuvant construct through optimized enzymatic conversion. The carbohydrate immunomodulatory anti-cancer adjuvant of the present invention modulates TME to re-educate tumor-associated macrophages to respond favorably to host immunity. Therefore, it was observed that the adjuvant treatment of the present invention induced a synergistic anti-tumor effect together with αPD-1 mAb. In other words, it was shown that the adjuvant of the present invention can enhance the immune response in immune-hyporesponsive tumors by activating innate immunity and leading to adaptive immunity. As modulation of the immune system has become increasingly important in cancer treatment, the adjuvant of the present invention provides a potential therapeutic strategy that can increase immunogenicity and improve the efficacy of T cell-based therapy.

While specific parts of the present invention have been described in detail above, it is apparent to those skilled in the art that such specific descriptions are merely preferred exemplary embodiments, and the scope of the present invention is not limited thereby. In other words, the substantial scope of the present invention is defined by the appended claims and their equivalents.

1. An adjuvant composition, comprising a galactose polymer which is interconnected by β-1,4 bonds.

2. The adjuvant composition of claim 1, wherein the galactose polymer is recognized by macrophages and induces TLR4-specific activation.

3. The adjuvant composition of claim 1, wherein the galactose polymer is specific for the innate immune signaling pathway.

4. The adjuvant composition of claim 1, wherein the galactose polymer reprograms M2-polarized macrophages to an M1-like phenotype in a TLR4-dependent manner.

5. The adjuvant composition of claim 1, wherein the galactose polymer changes the tumor microenvironment (TME) by reducing the number of helper T cells in the tumor.

6. The adjuvant composition of claim 1, wherein the galactose polymer is a polymer in which 4 to 20 galactose units are linked.

7. The adjuvant composition of claim 1, wherein the galactose polymer has a molecular weight of 100,000 to 200,000 kDa.

8. A vaccine composition, comprising the adjuvant composition according to claim 1.

9. The vaccine composition of claim 8, wherein the vaccine composition further comprises an antigen.

10. The vaccine composition of claim 9, wherein the antigen is selected from the group consisting of proteins, cells, viruses and combinations thereof.

11. A method for preparing an adjuvant composition, comprising the steps of:
extracting pectin heteropolysaccharide (PHP); and
sequentially treating the extracted pectin heteropolysaccharide (PHP) with enzymes.
